(19) United States
(12) Patent Application Publication
(10) Pub. No.: US 2026/0083431 A1
(43) Pub. Date: Mar. 26, 2026

(54) ULTRASOUND PROBE, SYSTEM, AND METHOD FOR MOVING A TRANSDUCER OF THE ULTRASOUND PROBE BETWEEN A NEUTRAL POSITION AND AN OFFSET POSITION BASED ON DETECTING A NON-USAGE TRIGGER OR A USAGE TRIGGER

(71) Applicant: GE Precision Healthcare LLC, Waukesha, WI (US)

(72) Inventors: Bernhard ASCHENBRENNER, Altmuenster (AT); Christian HEINRICH, Neukirchen (AT); Andreas MAIER, Lenzing (AT); Magali GEORGES, Biot (FR); Bruno HAIDER, Rehoboth Beach, DE (US)

(21) Appl. No.: 18/893,265

(22) Filed: Sep. 23, 2024

(51) Int. Cl. A61B 8/00

(52) U.S. Cl. CPC A61B 8/4209; A61B 8/467; A61B 8/54

(57) ABSTRACT

An ultrasound system may include an ultrasound probe including a transducer configured to acquire ultrasound data of a region of interest of a subject, and one or more processors configured to detect a non-usage trigger indicative of non-usage of the ultrasound probe for acquiring the ultrasound data of the region of interest of the subject, and control the transducer of the ultrasound probe to move from a neutral position to an offset position based on detecting the non-usage trigger.

### TECHNICAL FIELD

The present disclosure relates to an ultrasound probe including a transducer that is movable between a neutral position and an offset position based on detecting a non-usage trigger or a usage trigger. Further, the present disclosure relates to a system and method for detecting a usage trigger or a non-usage trigger, and controlling the transducer of the ultrasound probe between a neutral position and an offset position.

### BACKGROUND

An ultrasound system may generally include a console that houses various components of the ultrasound system and that supports a display for displaying ultrasound images. The ultrasound system may include one or more ultrasound probes that are respectively configured to connect to the console, and acquire ultrasound data of various anatomical regions of interest of a subject. For instance, the ultrasound system may include linear probes, phased probes, endocavitary probes, convex probes, etc.

An ultrasound probe may include a transducer that is configured to generate ultrasound signals, emit the ultrasound signals towards the region of interest of a subject, receive echo ultrasound signals that are back-scattered from the region of interest of the subject, and generate ultrasound data based on the echo ultrasound signals. An ultrasound system may generate an ultrasound image based on the echo ultrasound signals. The transducer may include various components, such as a lens, acoustic matching layer, piezoelectric layer, acoustic dematching layer, etc.

To perform ultrasound imaging, an operator may manipulate the ultrasound probe about an anatomical region of interest of a subject to acquire ultrasound data of the region of interest. After performing the ultrasound imaging, the operator may place the ultrasound probe in a probe holder of the console for storage. Alternatively, the operator may disconnect the ultrasound probe from the console, move the ultrasound probe to a storage area, connect the ultrasound probe to another console, perform a disinfection process, or the like. In some cases, the operator may move the console to another location.

During any of the foregoing situations, the ultrasound probe may be susceptible to external shock. For example, the operator may inadvertently drop the ultrasound probe while moving the ultrasound probe from the console to the subject, about the subject, from the subject to the console, or from the console to another area. Alternatively, the ultrasound probe may fall from the probe holder based on an external force, based on movement of the console, or the like. Alternatively, the ultrasound probe may inadvertently contact an external object. The sizes, structures, and materials of the components of the transducer, specifically, may render the transducer susceptible to damage in the event of external shock. Accordingly, an ultrasound probe may be rendered inoperable for ultrasound imaging after being exposed to an external shock due to damage to the transducer.

### SUMMARY

This summary introduces concepts that are described in more detail in the detailed description. It should not be used to identify essential features of the claimed subject matter, nor to limit the scope of the claimed subject matter.

According to an aspect, an ultrasound system may include an ultrasound probe including a transducer configured to acquire ultrasound data of a region of interest of a subject; and one or more processors configured to: detect a non-usage trigger indicative of non-usage of the ultrasound probe for acquiring the ultrasound data of the region of interest of the subject; and control the transducer of the ultrasound probe to move from a neutral position to an offset position based on detecting the non-usage trigger.

According to another aspect, an ultrasound probe may include a transducer configured to: acquire ultrasound data of a region of interest of a subject; move from a neutral position to an offset position based on a detection of a non-usage trigger indicative of non-usage of the ultrasound probe for acquiring the ultrasound data of the region of interest of the subject; and move from the offset position to the neutral position based on a detection of a usage trigger indicative of usage of the ultrasound probe for acquiring the ultrasound data of the region of interest of the subject.

According to another aspect, an ultrasound system may include a memory configured to store instructions; and one or more processors configured to execute the instructions to: detect a non-usage trigger indicative of non-usage of an ultrasound probe for acquiring ultrasound data of a region of interest of a subject; and control a transducer of the ultrasound probe to move from a neutral position to an offset position based on detecting the non-usage trigger.

### BRIEF DESCRIPTION OF DRAWINGS

FIG. 1 is a diagram of an example ultrasound system according to an embodiment.

FIG. 2 is a diagram of an example ultrasound system according to an embodiment.

FIG. 3 is a diagram of an example ultrasound probe according to an embodiment.

FIG. 4 is a diagram of an example ultrasound probe according to an embodiment.

FIG. 5 is a diagram of an example transducer of an ultrasound probe according to an embodiment.

FIG. 6 is a diagram of an example ultrasound probe according to an embodiment.

FIG. 7A is a diagram of an example transducer of an ultrasound probe in a neutral position according to an embodiment.

FIG. 7B is a diagram of an example transducer of an ultrasound probe in an offset position according to an embodiment.

FIG. 8 is a flowchart of an example process for controlling a transducer of an ultrasound probe to move between a neutral position and an offset position based on detecting a non-usage trigger.

FIG. 9 is a flowchart of an example process for controlling a transducer of an ultrasound probe to move between an offset position and a neutral position based on detecting a usage trigger.

### DETAILED DESCRIPTION

As addressed above, an ultrasound probe may be susceptible to damage in the event of an external shock. Specifically, the transducer of an ultrasound probe may be particularly susceptible given the location of the transducer in the ultrasound probe, and the propensity of the ultrasound probe to contact an external object or surface at the location of the transducer.

Some embodiments herein provide an ultrasound probe that includes a transducer that is movable between a neutral position and an offset position based on detecting a non-usage trigger or a usage trigger. Specifically, the transducer may be controlled to move to the offset position based on the detection of a non-usage trigger. The transducer may remain in the offset position until the transducer is controlled to move to the neutral position based on the detection of a usage trigger. In the offset position, the transducer may be less susceptible to damage in the event of an external shock than as compared to the transducer being in the neutral position. In this way, the ultrasound probe of the present disclosure is more robust than as compared to ultrasound probes that include a stationary transducer. Accordingly, the present disclosure provides an improved ultrasound probe and a technical improvement in the technical field of ultrasound imaging.

Further, the present disclosure may reduce, or remove, the need for a strict handling protocol of the ultrasound probe, which may improve the efficiency of non-scanning procedures, such as cleaning, disinfection, and/or handling. Further still, the present disclosure may permit the ultrasound probe to have a small form factor and/or may reduce the need to include various protective features, increased component size, or sensors in the ultrasound probe. For instance, in some cases, the ultrasound probe might not need a relatively thick external protective feature to provide protection for the lens. Such an external protective feature might increase the gap between the lens of the transducer and the subject, which can negatively impact image quality, increase signal attenuation, and increase reverberation. Accordingly, the ultrasound probe of the present disclosure may acquire ultrasound data having an improved image quality as compared to ultrasound probes with external protective features. Further, the ultrasound probe of the present disclosure may utilize a lens having a relatively smaller form factor, which can improve imaging quality as compared to ultrasound probes that implement thicker lenses. Further, the ultrasound probe might not require a sensor to detect a fall event. Such a sensor might have a high false positive rate and/or might increase the complexity of the ultrasound probe. By dispensing with the need for a sensor, the ultrasound probe of the present disclosure may reduce a number of situations resulting from false positives, and/or may be relatively less complex than ultrasound probes that include such sensors. Further still, the ultrasound probe of the present disclosure may maintain the transducer in the offset position until a usage trigger is detected. In this way, the ultrasound probe provides a protection mechanism that is applicable to additional external shock events in addition to fall events. Further still, the ultrasound probe may maintain the transducer in the offset position after the ultrasound probe is disconnected from the console and/or after the console is powered off. In this way, the ultrasound probe may increase the duration of maintaining protection of the transducer.

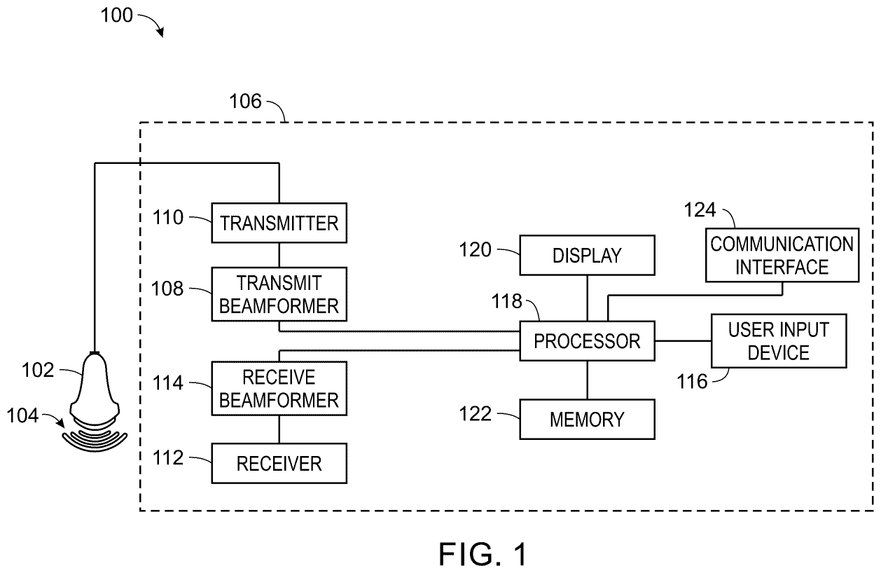

FIG. 1 is a diagram of an example ultrasound system 100 according to an embodiment. As shown in FIG. 1, the ultrasound system 100 may include an ultrasound probe 102, a transducer 104, a console 106, a transmit beamformer 108, a transmitter 110, a receiver 112, a receive beamformer 114, a user input device 116, a processor 118, a display 120, a memory 122, and a communication interface 124. The foregoing components may be connected via wired or wireless connections.

The ultrasound probe 102 may be configured to acquire ultrasound data for medical imaging, acquire ultrasound data for measuring blood flow, transmit ultrasound signals for tissue ablation, or the like. For example, the ultrasound probe 102 may be a linear probe, a phase array probe, a curved linear probe coupled with a position tracking system, a mechanically steered linear array transducer, an endocavitary probe, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 102 may be configured to generate ultrasound signals, emit the ultrasound signals towards the region of interest of a subject, receive echo ultrasound signals that are back-scattered from the region of interest of the subject, generate ultrasound data based on the echo ultrasound signals, and output the ultrasound data.

The console 106 may house the transmit beamformer 108, the transmitter 110, the receiver 112, the receive beamformer 114, the user input device 116, the processor 118, the display 120, the memory 122, and the communication interface 124. Additionally, the console 106 may be configured to connect to the ultrasound probe 102 to permit the transmitter 110 and the receiver 112 to communicate with the ultrasound probe 102.

The transmit beamformer 108 may be configured to apply delay times to electrical signals provided to the transducer 104 of the ultrasound probe 102 to focus corresponding ultrasound signals at the region of interest. The transmitter 110 may be configured to transmit electrical signals to the transducer 104 to drive the transducer 104 to emit ultrasound signals towards the region of interest. The transducer 104 may be configured to receive the electrical signals from the transmitter 110, convert the electrical signals into ultrasound signals, and emit the ultrasound signals towards the region of interest. The transducer 104 may be configured to receive echo ultrasound signals that are back-scattered by the region of interest, convert the echo ultrasound signals into electrical signals, and provide the electrical signals to the receiver 112. The receiver 112 may be configured to receive electrical signals from the elements, and provide the electrical signals to the receive beamformer 114. The receive beamformer 114 may apply delay times to the electrical signals received from the transducer 104.

The user input device 116 may be configured to receive a user input, and provide the user input to the processor 118. For example, the user input device 116 may be a user interface, a touch screen display, a keyboard, a keypad, a mouse, a button, a switch, a microphone, or the like. Additionally, or alternatively, the user input device 116 may be configured to sense information. For example, the user input device 116 may sense information from an electro-magnetic positioning system, an inertial measurement system, an accelerometer, a gyroscope, an actuator, or the like.

The processor 118 may be configured to perform the operations as described herein. For example, the processor 118 may be a central processing unit (CPU), a graphics processing unit (GPU), an accelerated processing unit (APU), a microprocessor, a controller, a microcontroller, a digital signal processor (DSP), a field-programmable gate array (FPGA), an application-specific integrated circuit (ASIC), or another type of processing component. The processor 118 may be implemented in hardware, firmware, or a combination of hardware and software. The processor 118 may include one or more processors 118 configured to perform the operations described herein. For example, a single processor 118 may be configured to perform all of the operations described herein. Alternatively, multiple processors 118, collectively, may be configured to perform all of the operations described herein, and each of the multiple processors 118 may be configured to perform a subset of the operations descried herein. For example, a first processor 118 may perform a first subset of the operations described herein, a second processor 118 may be configured to perform a second subset of the operations described herein, etc.

The processor 118 may be configured to control the ultrasound probe 102 to acquire ultrasound data. The processor 118 may be configured to control which of elements of the transducer 104 are active, and control the shape of a beam emitted from the transducer 104 of the ultrasound probe 102. The processor 118 may generate ultrasound images for display. For example, the processor 118 may generate B-mode images, color Doppler images, M-mode images, color M-mode images, or the like. The ultrasound images may be 4D images, 3D images, 2D images, single plane images, bi-plane images, three-plane images, multi-plane images, or the like. The ultrasound images may correspond to various anatomical planes (e.g., sagittal, coronal, and transverse) of the region of interest.

The display 120 may be configured to display information. For example, the display 120 may be a monitor, an LED display, a cathode ray tube, a projector display, a touchscreen, tablet computer, mobile phone, or the like. The display 120 may display ultrasound images based on the ultrasound data in real-time. For example, the display 120 may display the ultrasound images within one second, two seconds, five seconds, etc., of the ultrasound data being acquired by the ultrasound probe 102.

The memory 122 may be configured to store information and/or instructions for use by the processor 118. The memory 122 may be a non-transitory computer-readable medium. For example, the memory 122 may be a random access memory (RAM), a read only memory (ROM), and/or another type of dynamic or static storage device (e.g., a flash memory, a magnetic memory, and/or an optical memory) that stores information and/or instructions for use by the processor 118. The memory 122 may be configured to store instructions that, when executed by the processor 118, cause the processor 118 to perform the operations described herein.

The communication interface 124 may be configured to enable the processor 118 to communicate with other systems, such as via a wired connection, a wireless connection, or a combination of wired and wireless connections. For example, the communication interface 124 may include an Ethernet interface, an optical interface, a coaxial interface, an infrared interface, a radio frequency (RF) interface, a universal serial bus (USB) interface, a Wi-Fi interface, a cellular network interface, or the like.

The number and arrangement of the components of the ultrasound system 100 shown in FIG. 1 are provided as an example. In practice, the ultrasound system 100 may include additional components, fewer components, different components, or differently arranged components than those shown in FIG. 1. Additionally, or alternatively, a set of components (e.g., one or more components) of the ultrasound system 100 may perform one or more functions described as being performed by another set of components of the ultrasound system 100. Additionally, or alternatively, one or more components shown as being included in the console 106 may be provided in the ultrasound probe 102, and vice versa.

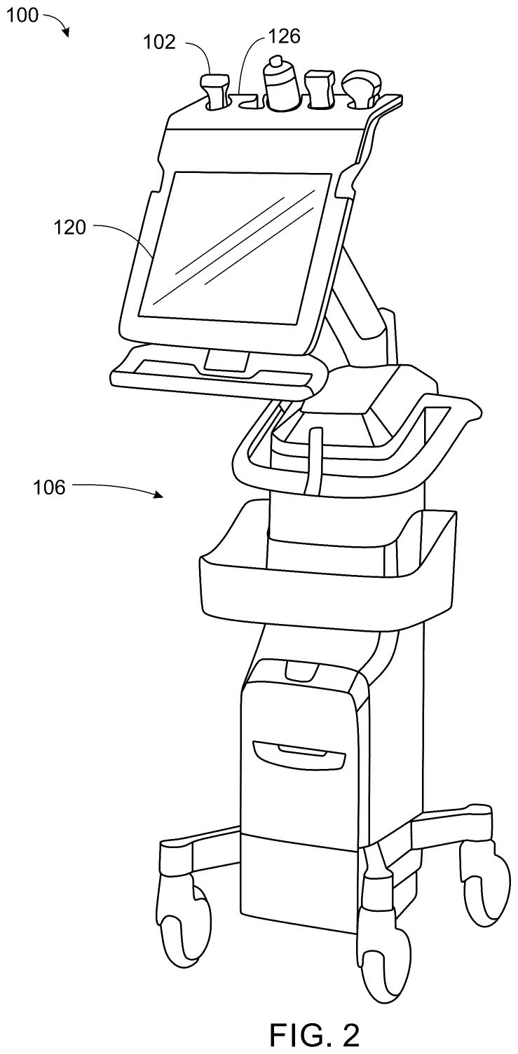

FIG. 2 is a diagram of an example ultrasound system 100 according to an embodiment. As shown in FIG. 2, the ultrasound system 100 may include the console 106, the display 120, and a probe holder 126. The probe holder 126 may be provided at a top of the display 120, and may include a plurality of slots that are each configured to hold and support an ultrasound probe 102.

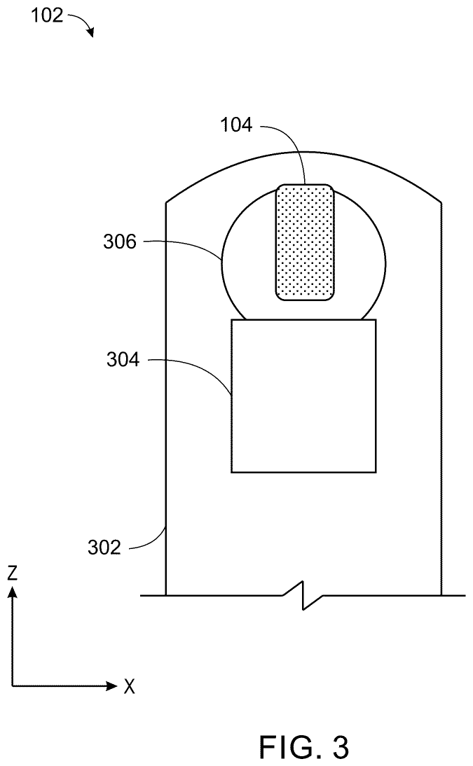

FIG. 3 is a diagram of an example ultrasound probe 102 according to an embodiment. As shown in FIG. 3, the ultrasound probe 102 may include a housing 302, a support 304, a rotational frame 306, and a transducer 104. The housing 302 may be configured to house the various components of the ultrasound probe 102. The support 304 may be configured to support the rotational frame 306, and permit the rotational frame to rotate about one or more axes. For instance, the rotational frame 306 may rotate about the z-axis as shown in FIG. 3, the x-axis as shown in FIG. 3, and/or the y-axis as shown in FIG. 3. The rotational frame 306 may be configured to rotate about one or more axes as shown in FIG. 3, and may be configured to support the transducer 104.

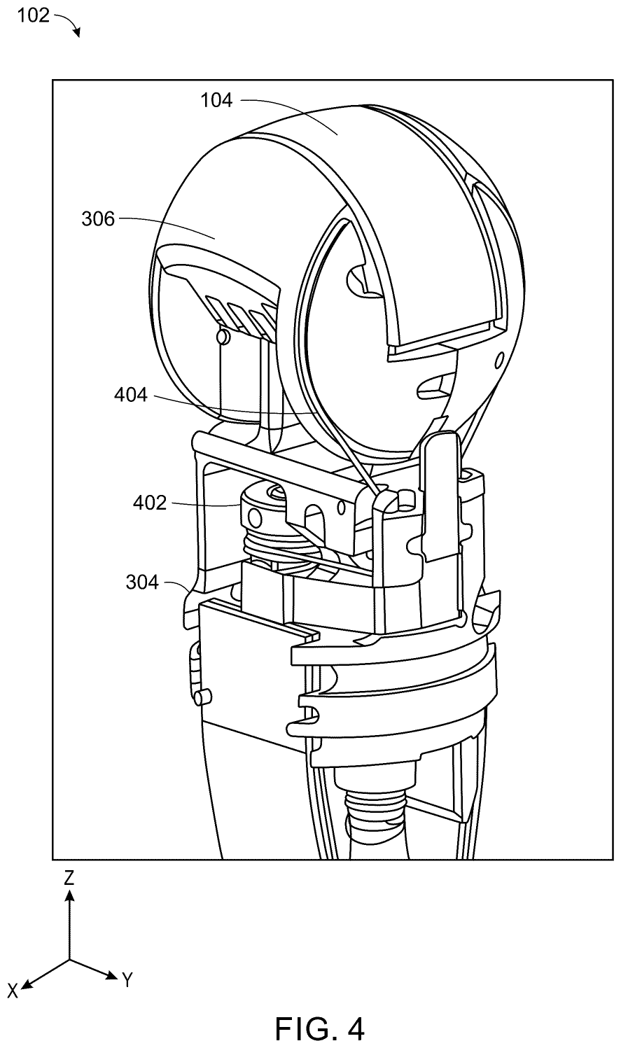

FIG. 4 is a diagram of an example ultrasound probe 102 according to an embodiment. As shown in FIG. 4, the ultrasound probe 102 may include a support 304, a rotational frame 306, a transducer 104, a rotational device 402, and a wire 404. The rotational device 402 may be connected to the rotational frame 306 via the wire 404. Based on movement of the rotational device 402, the rotational frame 306 may rotate about one or more axes as shown in FIG. 4.

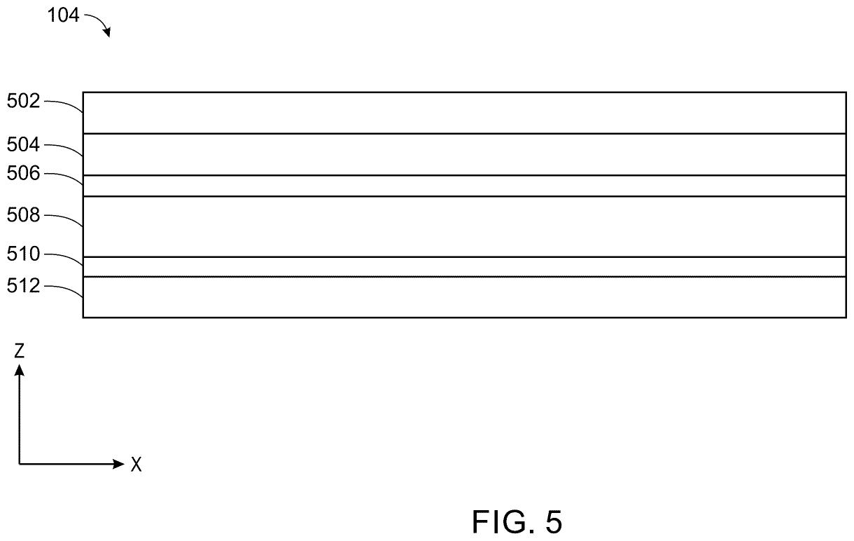

FIG. 5 is a diagram of an example transducer 104 of an ultrasound probe according to an embodiment. As shown in FIG. 5, the transducer 104 may include a lens 502, an acoustic matching layer 504, a first electrode 506, a piezoelectric layer 508, a second electrode 510, and an acoustic dematching layer 512. The lens 502 may be configured to direct and focus ultrasound signals towards a region of interest of a subject. For example, the lens 502 may be a polymer. The acoustic matching layer 504 may be configured to facilitate matching of an impedance differential that may exist between the relatively high impedance transducer 104 and the relatively low impedance subject. For example, the acoustic matching layer 504 may be graphite, a metal, or the like. The first electrode 506 may be a signal electrode, and the second electrode 510 may be a ground electrode that are configured to contact the piezoelectric layer 508 to transmit electrical signals. Alternatively, the first electrode 506 may be a ground electrode, and the second electrode 510 may be a signal electrode that are configured to contact the piezoelectric layer 508. For example, the first electrode 506 and/or the second electrode 510 may be gold, copper, nickel, silver, chromium, aluminum, or the like. The piezoelectric layer 508 may be configured to receive an electrical signal, deform based on the electrical signal, generate an ultrasound signal based on the deformation, and transmit the ultrasound signal towards a region of interest. Additionally, or alternatively, the piezoelectric layer 508 may be configured to receive an echo signal reflected by the region of interest, deform based on the echo signal, generate an electrical signal based on the deformation, and transmit the electrical signal. For example, the piezoelectric layer 508 may be Pb(Mg_{1/3}Nb_{2/3})O₃—PbTiO₃ (“PMN-PT”), Pb(In_{1/2}Nb_{1/2})O₃—Pb(Mg_{1/3}Nb_{2/3})O₃—PbTiO₃ (“PIN-PMN-PT”), Pb(ZrTi) (“PZT”), or the like. The piezoelectric layer 508 may include electrically-insulated regions that form elements of the ultrasound probe 102. The acoustic dematching layer 512 may be configured to decrease insertion losses and enhance a frequency bandwidth of the transducer 104. For example, the acoustic dematching layer 512 may be tungsten carbide, silicon carbide, or the like. Although particular components are shown in FIG. 5, it should be understood that the transducer 104 may include additional components, fewer components, or differently arranged components than as shown in FIG. 5. Additionally, the transducer 104 may employ other mechanisms than bulk piezoelectric mechanisms for the electromechanical conversion. For example, the transducer 104 may be constructed as a capacitive micromachined ultrasound transducer (cMUT), piezo micromachined ultrasound transducer (pMUT), or the like.

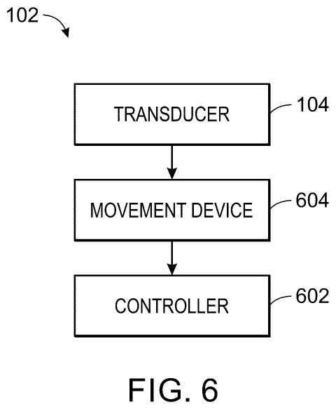

FIG. 6 is a diagram of an example ultrasound probe 102 according to an embodiment. As shown in FIG. 6, the ultrasound probe 102 may include a controller 602, a movement device 604, and a transducer 104. The controller 602 may be configured to receive a signal from the processor 118, and control the movement device 604 to move the transducer 104 between a neutral position and an offset position. For example, the controller 602 may be configured to receive a signal from the processor 118 based on the processor 118 detecting a non-usage trigger, and control the movement device 604 to move the transducer 104 from the neutral positon to the offset position. As another example, the controller 602 may be configured to receive a signal from the processor 118 based on the processor 118 detecting a usage trigger, and control the movement device 604 to move the transducer 104 from the offset position to the neutral position. The controller 602 may be a CPU, a GPU, an APU, a microprocessor, a microcontroller, a DSP, an FPGA, an ASIC, or another type of processing component. The movement device 604 may be configured to move the transducer between the neutral position and the offset position. For example, the movement device 604 may be a rotational device, a motor, an actuator, a spring, lever, or the like.

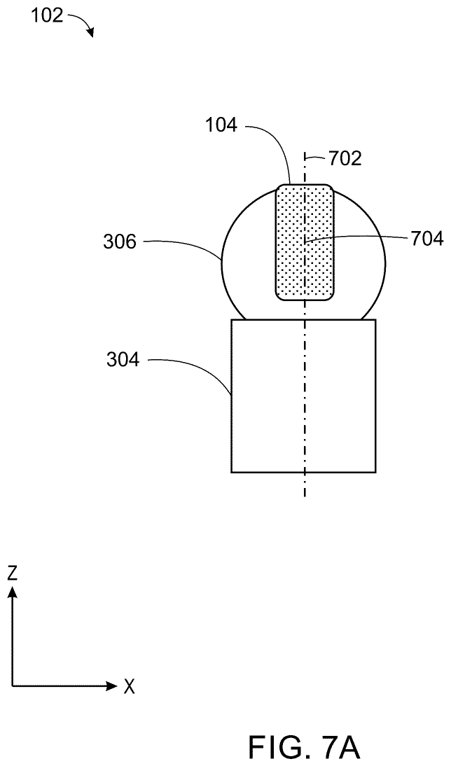

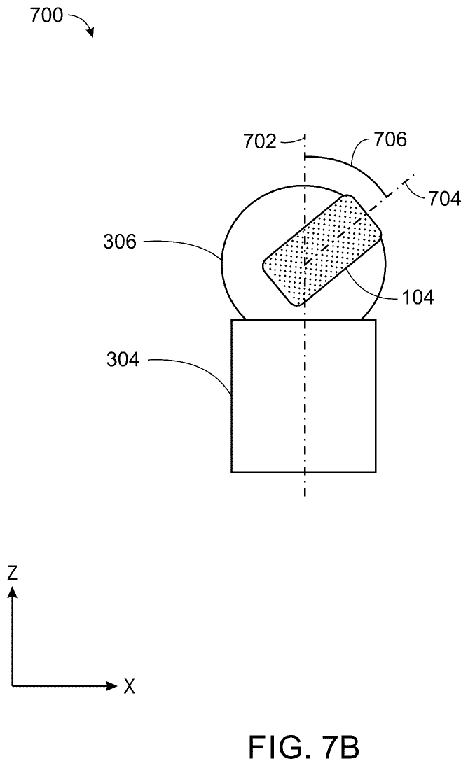

FIG. 7A is a diagram of an example transducer 104 of an ultrasound probe 102 in a neutral position according to an embodiment. As shown in FIG. 7A, an axis 702 may correspond to a longitudinal axis of the ultrasound probe 102, a longitudinal axis of the support 304, a longitudinal axis of the housing 302 of the ultrasound probe 102, or the like. Further, as shown in FIG. 7A, an axis 704 may correspond to a longitudinal axis of the transducer 104. The neutral position may be defined as a position in which the axis 702 and the axis 704 are substantially parallel. As used herein, “substantially parallel” may refer to the axis 702 and the axis 704 having 0° of offset, 2° of offset, 5° of offset, or the like.

FIG. 7B is a diagram of an example transducer 104 of an ultrasound probe 102 in an offset position according to an embodiment. As shown in FIG. 7B, the axis 702 may correspond to a longitudinal axis of the ultrasound probe 102, a longitudinal axis of the support 304, a longitudinal axis of the housing 302 of the ultrasound probe 102, or the like. Further, as shown in FIG. 7B, the axis 704 may correspond to a longitudinal axis of the transducer 104. The offset position may be defined as a position in which the axis 702 and the axis 704 are substantially offset. As used herein, “substantially offset” may refer to the axis 702 and the axis 704 being offset by 20°, 25°, 30°, 40°, 45°, 60°, 70°, 90°, 100°, or the like. Put another way, as shown, the axis 702 and the axis 704 may be offset by an angle 706. The angle 706 may be 20°, 25°, 30°, 40°, 45°, 60°, 70°, 90°, 100°, or the like. In this way, the rotational frame 306 may protect the transducer 104 in the event of an external shock to the ultrasound probe 102 because the rotational frame 306 is in a position where the transducer 104 would otherwise have been if the transducer 104 was still in the neutral position.

Although FIG. 7 depicts the axis 704 as being substantially parallel to a longitudinal axis of the ultrasound probe 102 in the neutral position, it should be understood that the axis 704 may be an axis having any predetermined position relative to the ultrasound probe 102 in the neutral position. In any event, the offset position may be a position that is offset from the neutral position regardless of the actual position of the neutral position.

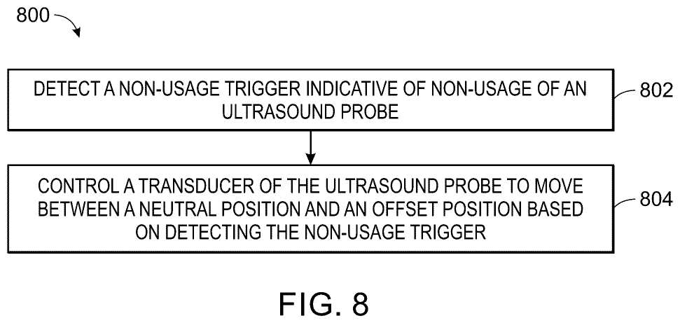

FIG. 8 is a flowchart of an example process 800 for controlling a transducer 104 of an ultrasound probe 102 to move between a neutral position and an offset position based on detecting a non-usage trigger. The operations of the process 800 may be performed by one or more components of the console 106. However, in other embodiments, one or more operations of the process 800 may be performed by one or more components of the ultrasound probe 102.

As shown in FIG. 8, the process 800 may include detecting a non-usage trigger indicative of non-usage of an ultrasound probe (operation 802). For example, the processor 118 may detect a non-usage trigger indicative of non-usage of the ultrasound probe 102. The non-usage trigger may be a trigger that is indicative of non-usage of the ultrasound probe 102. As used herein, “non-usage” may refer to the ultrasound probe 102 not being currently used for ultrasound imaging, intended to be not used for ultrasound imaging, or the like.

According to an embodiment, the non-usage trigger may be a user input via the user input device 116 that de-selects the ultrasound probe 102 for performing ultrasound imaging via the ultrasound system 100. For example, an operator may interact with the user input device 116 to input a user input that de-selects the ultrasound probe 102 to perform ultrasound imaging. The processor 118 may detect the non-usage trigger based on the user input.

Alternatively, the non-usage trigger may be a user input, via the user input device 116, of another ultrasound probe 102 that selects the other ultrasound probe 102 for performing ultrasound imaging via the ultrasound system 100. For example, the operator may interact with the user input device 116 to input a user input that selects another ultrasound probe 102 to perform ultrasound imaging. The processor 118 may detect the non-usage trigger based on the user input.

Alternatively, the non-usage trigger may be the positioning of the ultrasound probe 102 in a probe holder 126 of the ultrasound system 100. For example, the operator may position the ultrasound probe 102 in a probe holder 126 of the ultrasound system 100 after performing ultrasound imaging. The processor 118 may detect the non-usage trigger based on the positioning of the ultrasound probe 102 in the probe holder 126. For example, the processor 118 may detect the non-usage trigger based on a signal received from a sensor of the ultrasound system 100, based on tracking data received from an external tracking system, based on a signal received from the ultrasound probe 102, or the like, that identifies that the ultrasound probe 102 is positioned in the probe holder 126.

Alternatively, the non-usage trigger may be the absence of contact between a subject and the ultrasound probe 102. For example, the operator may remove the ultrasound probe 102 from contacting the subject after performing ultrasound imaging. The processor 118 may detect the non-usage trigger based on the absence of contact between the subject and the ultrasound probe 102. For example, the processor 118 may detect the non-usage trigger based on a signal received from a sensor of the ultrasound system 100, based on tracking data received from an external tracking system, based on a signal received from the ultrasound probe 102, or the like, that identifies that the ultrasound probe 102 is not contacting the subject.

Alternatively, the non-usage trigger may be the absence of contact between an operator of the ultrasound probe 102 and the ultrasound probe 102. For example, the operator may let go of the ultrasound probe 102 after performing ultrasound imaging. The processor 118 may detect the non-usage trigger based on the absence of contact between the operator and the ultrasound probe. For example, the processor 118 may detect the non-usage trigger based on a signal received from a sensor of the ultrasound system 100, based on tracking data received from an external tracking system, based on a signal received from the ultrasound probe 102, or the like, that identifies that the ultrasound probe 102 is not contacting the operator.

Alternatively, the non-usage trigger may be the absence of an ultrasound image displayed via the display 120. For example, the display 120 might not display any ultrasound images in the event that the ultrasound probe 102 is not within proximity to the subject. The processor 118 may detect the non-usage trigger based on the absence of an ultrasound image displayed via the display 120.

Alternatively, the non-usage trigger may be the absence of echo signals received via the ultrasound probe 102. For example, the ultrasound probe 102 might not receive any echo signals after the ultrasound probe 102 is no longer in proximity to the subject. The processor 118 may detect the non-usage trigger based on the absence of echo signals received via the ultrasound probe 102.

Alternatively, the non-usage trigger may be the generating of an ultrasound image that includes a quality metric that satisfies a threshold. For example, the ultrasound probe 102 may generate an ultrasound image that includes a quality metric (e.g., noise, sharpness, contrast, resolution, or the like) that satisfies a threshold indicative of low quality in the event that the ultrasound probe 102 is not within proximity to the subject. The processor 118 may detect the non-usage trigger based on the generating of the ultrasound image that includes a quality metric that satisfies the threshold.

Alternatively, the non-usage trigger may be a position of the ultrasound probe 102 relative to the ultrasound system 100. For example, the operator may position the ultrasound probe 102 in some position that is indicative of non-usage of the ultrasound probe 102 for ultrasound imaging after performing the ultrasound imaging. The processor 118 may detect the non-usage trigger based on the position of the ultrasound probe 102 relative to the ultrasound system 100. For example, the processor 118 may detect the non-usage trigger based on a signal received from a sensor of the ultrasound system 100, based on tracking data received from an external tracking system, based on a signal received from the ultrasound probe 102, or the like, that identifies a position of the ultrasound probe 102 relative to the ultrasound system 100.

Alternatively, the non-usage trigger may be a timeframe. For example, the timeframe may be an elapsed time since the beginning of the ultrasound imaging, an elapsed time since the ending of the ultrasound imaging, a time of day, a day of the week, or the like. The processor 118 may detect the non-usage trigger based on the timeframe.

Alternatively, the non-usage trigger may be a user input, via the user input device 116, that powers off the ultrasound system 100. For example, the operator may interact with the user input device 116 to power off the ultrasound system 100. The processor 118 may detect the non-usage trigger based on the user input.

Alternatively, the non-usage trigger may be the disconnection of the ultrasound probe 102 from the console 106. For example, the operator may disconnect the ultrasound probe 102 from the console 106. The processor 118 may detect the non-usage trigger based on the disconnection of the ultrasound probe 102 from the console 106.

Alternatively, the non-usage trigger may be a user input, via an input component of the ultrasound probe 102, that indicates that the ultrasound probe 102 is not to be used to perform ultrasound imaging. For example, the operator may interact with the input component of the ultrasound probe 102.

Alternatively, the non-usage trigger may be a drop event of the ultrasound probe 102. For example, the operator of the ultrasound probe 102 may inadvertently drop the ultrasound probe 102 causing a drop event. In this case, the processor 118 may detect the non-usage trigger based on the drop event. For example, the processor 118 may receive sensor data from a sensor of the ultrasound probe 102, and determine that the sensor data satisfies a threshold indicative of a drop event of the ultrasound probe 102. The sensor data may be acceleration data, rotation data, velocity data, or the like.

As further shown in FIG. 8, the process 800 may include controlling a transducer of the ultrasound probe to move between a neutral position and an offset position based on detecting the non-usage trigger (operation 804). For example, the processor 118 may control the transducer 104 of the ultrasound probe 102 to move from the neutral position to the offset position.

The neutral position may be a position in which an axis of the ultrasound probe 102 is substantially parallel to an axis of the transducer 104. For example, the neutral positon may be a positon in which the axes have 0°, 2°, 5°, or the like, of offset. Alternatively, the neutral position may be some predefined positon of the transducer 104. The offset position may be a position in which an axis of the ultrasound probe 102 is substantially offset from an axis of the transducer 104. For example, the offset position may be a position in which the axes have 20°, 25°, 30°, 40°, 45°, 60°, 70°, 90°, 100°, or the like, of offset. Additionally, or alternatively, the offset position may be a position that is substantially offset from the neutral position. For example, the offset position may be 20°, 25°, 30°, 40°, 45°, 60°, 70°, 90°, 100°, or the like, offset from the neutral position.

According to an embodiment, the processor 118 may send a signal to the controller 602 of the ultrasound probe 102 that causes the controller 602 to control the movement device 604 to move the transducer 104 from the neutral position to the offset position. Alternatively, the processor 118 may send a signal to the movement device 604 that causes the movement device 604 to move the transducer 104 from the neutral position to the offset position. Alternatively, the controller 602 may control the movement device 604 to move the transducer 104 from the neutral position to the offset position.

According to an embodiment, the ultrasound probe 102 may maintain the transducer 104 in the offset position until the detection of a usage trigger and/or the receiving of a signal that causes the transducer 104 to move from the offset position to the neutral position. The ultrasound probe 102 may maintain the transducer 104 in the offset position after the ultrasound probe 102 is disconnected from the console. Additionally, or alternatively, the ultrasound probe 102 may maintain the transducer 104 in the offset position after the ultrasound system 100 is powered off.

In this way, the rotational frame 306 may protect the transducer 104 in the event of an external shock to the ultrasound probe 102 because the rotational frame 306 is in a position where the transducer 104 would otherwise have been if the transducer 104 was still in the neutral position.

Although FIG. 8 depicts particular operations and a particular sequence of operations, it should be understood that other embodiments may include different operations and/or differently arranged operations than as shown in FIG. 8.

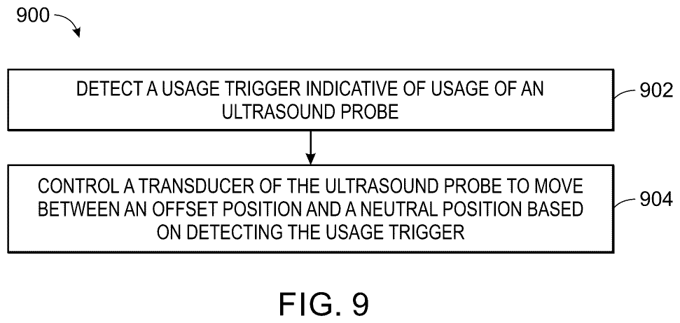

FIG. 9 is a flowchart of an example process 900 for controlling a transducer of an ultrasound probe to move between an offset position and a neutral position based on detecting a usage trigger. The operations of the process 900 may be performed by one or more components of the console 106. However, in other embodiments, one or more operations of the process 900 may be performed by one or more components of the ultrasound probe 102.

As shown in FIG. 9, the process 900 may include detecting a usage trigger indicative of usage of an ultrasound probe (operation 902). For example, the processor 118 may detect a usage trigger indicative of usage of the ultrasound probe 102. The usage trigger may be a trigger that is indicative of usage of the ultrasound probe 102. As used herein, “usage” may refer to the ultrasound probe 102 being currently used for ultrasound imaging, intended to be used for ultrasound imaging, or the like.

Alternatively, the usage trigger may be a user input, via the user input device 116, of the ultrasound probe 102 that selects the ultrasound probe 102 for performing ultrasound imaging via the ultrasound system 100. For example, the operator may interact with the user input device 116 to input a user input that selects the ultrasound probe 102 to perform ultrasound imaging. The processor 118 may detect the usage trigger based on the user input.

Alternatively, the usage trigger may be the removal of the ultrasound probe 102 from a probe holder 126 of the ultrasound system 100. For example, the operator may remove the ultrasound probe 102 from a probe holder 126 of the ultrasound system 100 before performing ultrasound imaging. The processor 110 may detect the usage trigger based on the removal of the ultrasound probe 102 from the probe holder 126. For example, the processor 118 may detect the usage trigger based on a signal received from a sensor of the ultrasound system 100, based on tracking data received from an external tracking system, based on a signal received from the ultrasound probe 102, or the like, that identifies that the ultrasound probe 102 has been removed from the probe holder 126.

Alternatively, the usage trigger may be the presence of contact between a subject and the ultrasound probe 102. For example, the operator may position the ultrasound probe 102 such that the ultrasound probe 102 contacts the subject before performing ultrasound imaging. The processor 118 may detect the usage trigger based on the presence of contact between the subject and the ultrasound probe 102. For example, the processor 118 may detect the usage trigger based on a signal received from a sensor of the ultrasound system 100, based on tracking data received from an external tracking system, based on a signal received from the ultrasound probe 102, or the like, that identifies that the ultrasound probe 102 is contacting the subject.

Alternatively, the usage trigger may be the presence of contact between an operator of the ultrasound probe 102 and the ultrasound probe 102. For example, the operator may contact the ultrasound probe 102 before performing ultrasound imaging. The processor 118 may detect the usage trigger based on the presence of contact between the operator and the ultrasound probe. For example, the processor 118 may detect the usage trigger based on a signal received from a sensor of the ultrasound system 100, based on tracking data received from an external tracking system, based on a signal received from the ultrasound probe 102, or the like, that identifies that the ultrasound probe 102 is contacting the operator.

Alternatively, the usage trigger may be a position of the ultrasound probe 102 relative to the ultrasound system 100. For example, the operator may position the ultrasound probe 102 in some position that is indicative of usage of the ultrasound probe 102 for ultrasound imaging before performing the ultrasound imaging. The processor 118 may detect the usage trigger based on the position of the ultrasound probe 102 relative to the ultrasound system 100. For example, the processor 118 may detect the usage trigger based on a signal received from a sensor of the ultrasound system 100, based on tracking data received from an external tracking system, based on a signal received from the ultrasound probe 102, or the like, that identifies a position of the ultrasound probe 102 relative to the ultrasound system 100.

Alternatively, the usage trigger may be a timeframe. For example, the timeframe may be a time of a scheduled scan of the subject, a time of day, a day of the week, or the like. The processor 118 may detect the usage trigger based on the timeframe.

Alternatively, the usage trigger may be a user input, via the user input device 116, that powers on the ultrasound system 100. For example, the operator may interact with the user input device 116 to power on the ultrasound system 100. The processor 118 may detect the usage trigger based on the user input.

Alternatively, the usage trigger may be a user input, via an input component of the ultrasound probe 102, that indicates that the ultrasound probe 102 is to be used to perform ultrasound imaging. For example, the operator may interact with the input component of the ultrasound probe 102.

As further shown in FIG. 9, the process 900 may include controlling a transducer of the ultrasound probe to move between an offset position and a neutral position based on detecting the usage trigger (operation 904).

According to an embodiment, the processor 118 may send a signal to the controller 602 of the ultrasound probe 102 that causes the controller 602 to control the movement device 604 to move the transducer 104 from the offset position to the neutral position. Alternatively, the processor 118 may send a signal to the movement device 604 that causes the movement device 604 to move the transducer 104 from the offset position to the neutral position. Alternatively, the controller 602 may control the movement device 604 to move the transducer 104 from the offset position to the neutral position.

According to an embodiment, the ultrasound probe 102 may maintain the transducer 104 in the neutral position until the detection of a non-usage trigger and/or the receiving of a signal that causes the transducer 104 to move from the neutral position to the offset position.

Although FIG. 9 depicts particular operations and a particular sequence of operations, it should be understood that other embodiments may include different operations and/or differently arranged operations than as shown in FIG. 9.

In this way, some embodiments herein provide an ultrasound probe that includes a transducer that is movable between a neutral position and an offset position based on detecting a non-usage trigger or a usage trigger. Specifically, the transducer may be controlled to move to the offset position based on the detection of a non-usage trigger. The transducer may remain in the offset position until the transducer is controlled to move to the neutral position based on the detection of a usage trigger. In the offset position, the transducer may be less susceptible to damage in the event of an external shock than as compared to the transducer being in the neutral position. In this way, the ultrasound probe of the present disclosure is more robust than as compared to ultrasound probes that include a stationary transducer. Accordingly, the present disclosure provides an improved ultrasound probe and a technical improvement in the technical field of ultrasound imaging.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. An ultrasound system comprising:
an ultrasound probe including a transducer configured to acquire ultrasound data of a region of interest of a subject; and
one or more processors configured to:
detect a non-usage trigger indicative of non-usage of the ultrasound probe for acquiring the ultrasound data of the region of interest of the subject; and
control the transducer of the ultrasound probe to move from a neutral position to an offset position based on detecting the non-usage trigger.

2. The ultrasound system of claim 1, wherein, in the neutral position, a longitudinal axis of the transducer is substantially parallel to a longitudinal axis of the ultrasound probe.

3. The ultrasound system of claim 1, wherein, in the offset position, a longitudinal axis of the transducer is substantially offset from a longitudinal axis of the ultrasound probe.

4. The ultrasound system of claim 1, wherein the non-usage trigger is a user input, via a user interface, that de-selects the ultrasound probe for performing ultrasound imaging via the ultrasound system.

5. The ultrasound system of claim 1, wherein the non-usage trigger is a user input, via a user interface, of another ultrasound probe for performing ultrasound imaging via the ultrasound system.

6. The ultrasound system of claim 1, wherein the non-usage trigger is a positioning of the ultrasound probe in a probe holder of the ultrasound system.

7. The ultrasound system of claim 1, wherein the non-usage trigger is an absence of contact between the subject and the ultrasound probe.

8. The ultrasound system of claim 1, wherein the non-usage trigger is an absence of an ultrasound image displayed via a display of the ultrasound system.

9. The ultrasound system of claim 1, wherein the non-usage trigger is a generating of an ultrasound image that includes a quality metric that satisfies a threshold.

10. The ultrasound system of claim 1, wherein the non-usage trigger is a position of the ultrasound probe relative to the ultrasound system.

11. The ultrasound system of claim 1, wherein the non-usage trigger is a user input, via a user interface, that powers off the ultrasound system.

12. The ultrasound system of claim 1, wherein the ultrasound probe is configured to maintain the transducer in the offset position after the ultrasound probe is disconnected from a console of the ultrasound system.

13. The ultrasound system of claim 1, wherein the ultrasound probe is configured to maintain the transducer in the offset position after the ultrasound system is powered off.

14. The ultrasound system of claim 1, wherein the one or more processors are further configured to:
detect a usage trigger indicative of usage of the ultrasound probe for acquiring the ultrasound data of the region of interest of the subject; and
control the transducer of the ultrasound probe to move from the offset position to the neutral position based on detecting the usage trigger.

15. An ultrasound probe comprising:
a transducer configured to:
acquire ultrasound data of a region of interest of a subject;
move from a neutral position to an offset position based on a detection of a non-usage trigger indicative of non-usage of the ultrasound probe for acquiring the ultrasound data of the region of interest of the subject; and
move from the offset position to the neutral position based on a detection of a usage trigger indicative of usage of the ultrasound probe for acquiring the ultrasound data of the region of interest of the subject.

16. The ultrasound probe of claim 15, wherein, in the neutral position, a longitudinal axis of the transducer is substantially parallel to a longitudinal axis of the ultrasound probe.

17. The ultrasound probe of claim 15, wherein, in the offset position, a longitudinal axis of the transducer is substantially offset from a longitudinal axis of the ultrasound probe.

18. The ultrasound probe of claim 15, further comprising:
a movement device configured to move the transducer from the neutral position to the offset position.

19. The ultrasound probe of claim 15, wherein the ultrasound probe is configured to maintain the transducer in the offset position after the ultrasound probe is disconnected from a console of an ultrasound system or after the ultrasound system is powered off.

20. An ultrasound system comprising:
a memory configured to store instructions; and
one or more processors configured to execute the instructions to:
detect a non-usage trigger indicative of non-usage of an ultrasound probe for acquiring ultrasound data of a region of interest of a subject; and
control a transducer of the ultrasound probe to move from a neutral position to an offset position based on detecting the non-usage trigger.
